(19) United States
(12) Patent Application Publication
(10) Pub. No.: US 2007/0149766 A1
(43) Pub. Date: Jun. 28, 2007

(54) Method for producing a recombinant protein of interest and protein thus produced

(75) Inventors: Bernard Mouillac, Montpellier (FR); Jean-Louis Baneres, Montpellier (FR); Tuhinadri Sen, Kolkata (IN)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: INSERM, PARIS CEDEX 16, FRANCE (FR)

(21) Appl. No.: 10/561,107

(22) PCT Filed: Jun. 18, 2004
(86) PCT No.: PCT/FR04/01538

§ 371 Date: Feb. 14, 2006

(30) Foreign Application Priority Data
Jun. 19, 2003 (FR) 03/07411

(51) Int. Cl. C07K 14/705

(52) U.S. Cl. 530/350

(57) ABSTRACT

A fragment of an alpha-integrin for producing at least one recombinant protein of interest in a cell, with the exception of a mammalian cell.

### RELATED APPLICATION

This is a §371 of International Application No. PCT/FR2004/001538, with an international filing date of Jun. 18, 2004 (WO 2004/113539, published Dec. 29, 2004), which is based on French Patent Application No. 03/07411, filed Jun. 19, 2003.

### FIELD OF THE INVENTION

This invention relates to a method of producing large quantities of a protein of interest which can be used directly for structural analyses. The invention also relates to the recombinant protein obtained.

### BACKGROUND

G protein-coupled receptors (GPCRS) constitute a superfamily of membrane proteins characterized by 7 transmembrane domains (TM I to VII) which play an essential role in intercellular communication and the reception of sensory signals (1).

With several hundred members identified, GPCRs form the largest structural and functional family of membrane receptors. They represent in particular a significant part of the human genome known to date (at least 700 receptors, 0.5% of the genome).

Expressed at the surface of all the cells of an organism (from yeast to humans), they are activated by a large variety of extracellular messages (peptides, hormones, lipids, odorous molecules, light, nucleotides, nucleosides, taste molecules, etc.). Activation thereof gives rise to an intracellular cascade of signals via G proteins and results in a large number of cellular responses (for example, cell division or shrinkage, neurotransmission).

In general, GPCRs are involved in each physiological function. The importance of these receptors and the fact that their location in the cell is known makes them ideal targets for therapy. In fact, it may be estimated that almost 50% of medicaments on the market act via GPCRs. Many pathologies are the result of GPCR mutations and their clinical manifestations are well known. Mention may be made, for example, of blindness, nephrogenic diabetes insipidus, hypothyroidism or hyperthyroidism, precocious puberty, obesity (2).

The discovery that some chemokine receptors are cofactors of infection by the HIV virus reinforces the idea that GPCRs are involved in a wide range of pathological situations (3).

These general considerations demonstrate the need to study the functional architecture of these receptors to better understand the signal transduction process and the dynamics of their interactions with various molecules (ligands or intracellular partners), and to develop new pharmacological and therapeutic tools. However, the study of the functional architecture of GPCRs using “direct” experimental methods (X-ray crystallography, NMR, mass spectrometry) still remains very limited. Just one three-dimensional (3D) structure is currently known, that of bovine rhodopsin (4), on account of the very high natural level of expression of this receptor in the retina. Knowledge of their functional architecture is thus currently obtained using a set of methods involving theoretical methods (modeling), physicochemical methods (photolabeling, fluorescence) and biological methods (site-specific mutagenesis, molecular pharmacology, knock-out, etc.).

Those studies are of extreme importance on the industrial and socioeconomic levels, given the potential therapeutic applications.

However, studying the structure and function of GPCRs is very difficult for various reasons:
the transmembrane nature of these proteins and their hydrophobicity makes them delicate to handle and usually leads to a loss of functionality and to denaturation following solubilization;
it remains very difficult to obtain them in their complete primary sequence. Most of the time, they are expressed in truncated form (5);
they are expressed in very low quantity (0.01% of membrane proteins), which forms an obstacle to purifying them in large quantities;
their molecular weight is high (greater than 40 kD), and they are characterized by the presence of post-translational modifications (glycosylation, palmitoylation, phosphorylation) and particular structural features (disulfide bridges);
they are multifunctional proteins having domains with different roles: ligand binding, G protein activation, allosteric sites, zones involved in their regulation/desensitization.

It will be easily understood that the critical step which at present forms a real obstacle is that of obtaining GPCRs in amounts compatible with “direct” structural biology approaches.

To date, no strategy has been developed for producing them in large quantity and in a way which can be generalized to all GPCRs, and which furthermore allows simple purification thereof in a functional form. At times, some receptors have been produced in high quantities (mg/l of culture) (6-8), but the methods used cannot be applied to most GPCRs.

Within the context of producing a protein in large quantities, GPCRs represent only one example of the difficulties encountered when trying to obtain a large quantity of a protein of interest. It would therefore be advantageous to provide a method for producing a large quantity of a protein of interest, particularly GPCRs.

### SUMMARY OF THE INVENTION

This invention relates to a fragment of an alpha-integrin for producing at least one recombinant protein of interest in a cell, with the exception of a mammalian cell.

This invention also relates to a recombinant protein including at least one fragment of the alpha-integrin and at least one membrane protein of interest.

This invention further relates to a nucleotide sequence coding for at least one recombinant protein of interest.

This invention still further relates to a vector including the nucleotide sequence.

This invention also further relates to a cell, with the exception of a mammalian cell, into which the nucleotide sequence or the vector has been introduced.

This invention further yet relates to a method for producing at least one protein of interest including introducing into a cell, with the exception of a mammalian cell, the nucleotide sequence coding for at least one recombinant protein, and placing the cell under conditions which allow expression of the recombinant protein(s) of interest.

### BRIEF DESCRIPTION OF THE DRAWINGS

FIG. 1 shows a construct corresponding to a vector according to aspects of the invention.

FIG. 2 shows production of the α5-integrin/vasopressin V2 receptor fusion protein according to the method of the invention (left-hand column: molecular weight of the proteins of the marker sample; arrow: position of the α5-integrin/vasopressin V2 receptor recombinant protein, NI: proteins of a non-induced sample, 2h, 3h and 4h: proteins of an induced sample after 2h, 3h and 4h of induction).

FIG. 3 shows the α5-integrin/vasopressin V2 receptor recombinant protein of FIG. 2 after purification and migration on electrophoresis gel (left-hand column: molecular weight of the proteins of the marker sample; arrow: position of the α5-integrin/vasopressin V2 receptor recombinant protein).

FIG. 4 shows the result of purification of the α5-integrin/vasopressin V2 receptor CXCR4 (α5-V2-CXCR4) recombinant fusion protein by means of affinity chromatography.

S6M: supernatant of solubilization in 6M urea buffer, deposited on Ni-NTA agarose resin

FT: sample not held on the resin

W: fraction wash containing 15 mM imidazole

E100: purified fusion eluted in a buffer containing 100 mM imidazole

The arrow indicates the position of the α5-V2-CXCR4 fusion protein.

### DETAILED DESCRIPTION

We have surprisingly demonstrated that the construct of recombinant proteins, particularly membrane proteins, and most particularly GPCRs, comprising at least one fragment of an alpha-integrin and the protein of interest, makes it possible to obtain recombinant proteins capable of being expressed in large quantities. This strategy makes it possible in particular to obtain a production of the proteins in a large quantity in microorganisms, particularly in bacteria. When the recombinant proteins are produced in bacteria, they accumulate in the inclusion body of the bacterial cytoplasm. It is then necessary to renaturate the proteins of interest to obtain them in active form in a quantity compatible with direct analysis of their structure, for example, by X-ray crystallography or nuclear magnetic resonance (NMR). The method can furthermore permit production of non-truncated proteins, particularly when it is applied to GPCRs.

Integrins form a family of receptors which are associated in terms of their structure and function and which participate in cell-cell and cell-extracellular matrix interactions. All the integrins are in the form of heterodimers of alpha and beta subunits, which are bonded noncovalently. Based on their primary sequence, all the alpha-integrins have an N-terminal region formed of seven repeated amino acid sequences (repeats I to VII), each comprising approximately 60 amino acids. Some alpha subunits include an insertion domain (I domain) of around 200 amino acids, located between repeats II and III. The homologies between repeats I and VII essentially comprise consensus sequences FG and GAP, corresponding to the phenylalanine, glycyl-glycyl, alanyl and prolyl chains, hence their name “FG-GAP repeat”.

We are not aware that the alpha subunit of integrins (also referred to as alpha-integrin (α-integrin)) has been used to produce recombinant proteins of interest in cells other than mammalian cells, and to do so in a quantity that is directly compatible with structural analysis of the protein of interest, this requiring a quantity of the protein which may be as much as several milligrams.

Thus, one aspect of the invention relates to the use of at least one fragment of an alpha-integrin in the construct of at least one recombinant protein of interest. It also relates to the use of at least one fragment of an alpha-integrin for producing at least one recombinant protein of interest.

The expression “recombinant protein” or “recombinant protein of interest” as used herein relates to the recombinant protein produced according to aspects of the invention. This recombinant protein may in particular comprise the chaining of several (at least two) proteins of interest which are fused, and which may optionally be separated by spacer sequences and/or cleavage sequences.

The expression “protein of interest” relates to the peptide sequence corresponding to a protein of interest which it is desired to produce (or which has been produced).

Thus, it will be understood that a “recombinant protein” is formed of one or more “proteins of interest”, optionally separated by spacer sequences and/or cleavage sequences.

“Fragment of an alpha-integrin” will be understood to mean both the complete amino acid sequence of the alpha-integrin used and also a partial sequence. The sequence of the alpha-integrin which is used may be native or mutated. Preferably, the sequence used is a sequence comprising the N-terminal end of the alpha-integrin used, even more preferably a sequence corresponding to the N-terminal end of the alpha-integrin used.

The fragment of the alpha-integrin used may comprise at least FG-GAP modules IV to VII and a portion of FG-GAP module III of the alpha-integrin used.

The fragment of the alpha-integrin used may be a fragment of any known alpha-integrin. Mention will be made in particular of the integrins α1, α2, α3, α4, α5, α6, α7, α8, α9, α10, α11, αD, αE, αL, αM, αX, αIIb or αV.

Use may be made of a fragment of 287 amino acids, corresponding to the part of the N-terminal end of alpha-5-integrin which extends between positions 231 and 517, according to the numbering which takes account of the presence of the signal peptide. If account is not taken of the signal peptide, the fragment which can be used extends from position 190 (G residue) to 476 (G residue) of alpha-5-integrin.

When use is made of other alpha-integrins, the fragments which can be used are the fragments homologous to the fragments defined above. For example, in the case of αV-integrin, the fragment which can be used corresponds to the part of the N-terminal end of αV-integrin which extends from position 211 (G residue) to 495 (G residue) according to the numbering which takes account of the presence of the signal peptide. If account is not taken of the signal peptide, the fragment which can be used extends from position 181 (G residue) to 465 (G residue) of αV-integrin. In the case of αIIb-integrin, the fragment which can be used corresponds to the part of the N-terminal end of αIIb-integrin which extends from position 224 (G residue) to 508 (Q residue) according to the numbering which takes account of the presence of the signal peptide. If account is not taken of the signal peptide, the fragment which can be used extends from position 193 (G residue) to 477 (Q residue) of αIIb-integrin.

The fragment of the alpha-integrin used may comprise at least one amino acid sequence selected from the sequences SEQ ID No. 1 (fragment of human α5-integrin), SEQ ID No. 2 (fragment of human V-integrin) and SEQ ID No. 3 (fragment of human αIIb-integrin) in the appended sequence listing.

The alpha-integrin fragment used may comprise at least one amino acid sequence encoded by one of the nucleotide sequences selected from the sequences SEQ ID No. 4 (fragment of human α5-integrin), SEQ ID No. 5 (fragment of human V-integrin) and SEQ ID No. 6 (fragment of human αIIb-integrin) in the appended sequence listing.

It is possible that the fragment of alpha-integrin is used in the construct of several (at least two) recombinant proteins of interest. In this case, the recombinant proteins will be fused during translation. This may prove necessary in the case of a protein of interest in respect of which the construct does not allow its direct production (refractory protein). It is then necessary to couple in tandem the sequence of the refractory protein to a recombinant protein of interest which the constructs make it possible to produce. Thus, the construct may comprise at least one DNA fragment encoding at least one fragment of an alpha-integrin, then at least one DNA encoding at least a first recombinant protein of interest and at least one DNA encoding at least a second recombinant protein of interest. The DNA encoding the second protein of interest may be inserted in the construct in phase downstream of the DNA sequence encoding the first protein of interest. This can be combined with any one of the aspects described above.

Preferably, the alpha-integrin fragment is located in the recombinant protein of interest upstream of the sequence of the protein of interest (or proteins of interest) to be produced, that is to say at the N-terminal end of the recombinant protein of interest (or recombinant proteins of interest) which are to be constructed and/or produced.

Aspects of the invention also relate to a recombinant protein, comprising, fused together, at least one fragment of an alpha-integrin as defined above and at least one protein of interest.

The protein(s) of interest, which forms (form) part of the recombinant protein, may be any protein which it is desired to produce, particularly a membrane protein, more particularly a G protein-coupled receptor (GPCRs). By way of example of the latter, mention may be made of vasopressin and oxytocin receptors (V1a, V2, OTR), leukotriene receptors (BLT1, BLT2, CysLT1, CysLT2), adrenergic receptors (beta-3), cannabinoid receptors (CB1), chemokine receptors (CCR5, CXCR4), the angiotensin II AT1 receptor, the bradykinin B2 receptor.

The recombinant protein (regardless of the embodiment of the invention) may further comprise any amino acid sequence which makes it possible to purify the protein in a simple manner. Thus, the recombinant protein may comprise a sequence of 6 histidine residues (6×HIS tag; SEQ ID NO: 12). This 6×HIS (SEQ ID NO: 12) tag may be incorporated in the sequence of the protein with a view to its purification on a Ni-NTA (nickel-nitrilotriacetic acid) agarose column. Preferably, this sequence is at the C-terminal end of the recombinant protein. When the recombinant protein consists of at least two fused proteins, the 6×HIS tag (SEQ ID NO: 12) is preferably located downstream of the last of the proteins of interest which it is desired to produce.

The sequence encoding the recombinant protein may further comprise at least one sequence encoding at least one endoprotease cleavage site.

Advantageously, the sequence coding for the last residues of the integrin may be mutated to form an endoprotease cleavage site (factor Xa, thrombin), which, following expression and purification of the recombinant protein, will make it possible to separate the protein of interest from its fusion partner. The L residue (position 285) may be modified by mutation into an I residue, the E and G residues (positions 286 and 287) being preserved. An additional R residue may be introduced by mutagenesis. The chain thus formed (IEGR; SEQ ID NO: 9) corresponds to the factor Xa cleavage site which cuts the protein after the R residue.

The factor Xa cleavage site can be transformed into a thrombin cleavage site. To do this, the I, E and G residues can be replaced by L, V and P residues. The R residue is preserved to obtain the chain LVPR (SEQ ID NO: 10). Since the integrin fragment has been incorporated into the vector at the 3′ end by a BamHI site (sequence ggatcc), there is thus obtained the sequence ggatcc coding for two residues G and S just after LVPR (SEQ ID NO: 10). The LVPRGS (SEQ ID NO: 11) chain forms the thrombin cleavage site, which cuts the protein after the R residue.

It will thus be understood that the recombinant protein may comprise, from its N-terminal end to its C-terminal end, the alpha-integrin fragment comprising the endoprotease cleavage site, the protein(s) of interest and the 6×HIS tag (SEQ ID NO: 12).

The recombinant protein may comprise, from its N-terminal end to its C-terminal end, the alpha-integrin fragment comprising the factor Xa cleavage site, the protein(s) of interest and the 6×HIS tag (SEQ ID NO: 12).

The recombinant protein may comprise, from its N-terminal end to its C-terminal end, the alpha-integrin fragment comprising the thrombin cleavage site, the protein(s) of interest and the 6×HIS tag (SEQ ID NO: 12).

It may still be necessary, when the recombinant protein comprises more than one protein of interest which are fused together, that the proteins of interest can be separated after synthesis, for example, before purification. Thus, it is possible to insert, between the different DNA sequences encoding the different proteins of interest, at least one DNA sequence encoding an endoprotease cleavage site. It is possible for cleavage sites for different endonucleases to be inserted into the same recombinant protein.

It may be necessary to make the cleavage of the recombinant protein even more effective. In this respect, it is possible to insert into the construct a sequence encoding a peptide sequence which serves as a spacer arm, preferably located upstream of the endoprotease cleavage site.

Thus, the recombinant protein may further comprise a peptide sequence serving as a spacer arm, preferably located upstream of the endoprotease cleavage site.

Therefore, the recombinant protein may comprise, from its N-terminal end to its C-terminal end, the alpha-integrin fragment comprising a spacer arm and the endoprotease cleavage site, the protein(s) of interest and the 6×HIS tag (SEQ ID NO: 12).

The recombinant protein may comprise, from its N-terminal end to its C-terminal end, the alpha-integrin fragment comprising a spacer arm, the factor Xa cleavage site, the protein(s) of interest and the 6×HIS tag (SEQ ID NO: 12).

The recombinant protein may also comprise, from its N-terminal end to its C-terminal end, the alpha-integrin fragment comprising a spacer arm, the thrombin cleavage site, the protein(s) of interest and the 6×HIS tag (SEQ ID NO: 12).

The sequence encoding a peptide sequence which serves as a spacer arm may be any sequence known in the art which allows a sufficient spacing between the endoprotease cleavage site and the protein(s) of interest for the cleavage of the recombinant protein to be effective.

Preferably, the sequence encoding a peptide sequence serving as a spacer arm is the following sequence SEQ ID No. 7: 5′GACCCGGGTGGTGGTGGTGGTGGTGGTGGTGGT 3′ encoding the following peptide sequence SEQ ID No. 8: DPGGGGGGGG.

Thus, the recombinant protein may comprise, from its N-terminal end to its C-terminal end, the alpha-integrin fragment comprising a spacer arm and the endoprotease cleavage site, the protein(s) of interest, separated by one or more endoprotease cleavage sites (for example, factor Xa or thrombin cleavage sites) and the 6×HIS tag (SEQ ID NO: 12).

Aspects of the invention also relate to the use of at least one fragment of a nucleotide sequence coding for at least one fragment of an alpha-integrin as defined above, in the construct of a nucleotide sequence coding for a recombinant protein of interest as defined above.

Aspects of the invention further relate to the use of at least one fragment of a nucleotide sequence coding for at least one fragment of an alpha-integrin as defined above, for producing a recombinant protein of interest as defined above.

Aspects of the invention still further relate to a nucleotide sequence coding for a recombinant protein of interest comprising at least one fragment of a nucleotide sequence coding for at least one fragment of an alpha-integrin, as defined above, and a nucleotide sequence coding for at least one protein of interest, as defined above.

Preferably, the nucleotide sequence coding for at least one fragment of an alpha-integrin which can be used or is included in the nucleotide sequence coding for a recombinant protein of interest and may be selected from the nucleotide sequences SEQ ID No. 4, SEQ ID No. 5 and SEQ ID No. 6 in the appended sequence listing.

Aspects of the invention yet further relate to a vector comprising a nucleotide sequence coding for a recombinant protein of interest, as defined above, comprising at least one fragment of a nucleotide sequence coding for at least one fragment of an alpha-integrin and a nucleotide sequence coding for at least one protein of interest. The vector may be a eukaryotic vector such as a plasmid or a virus. The vector may also be any prokaryotic vector such as a plasmid or a phage.

Preferably, the vector is an expression vector, that is to say a vector capable of allowing the transcription and translation of the nucleotide sequence it contains.

By way of example, mention may be made of the vectors of the pET family which are sold by the company Novagen or those of the pGEX family which are sold by the company Amersham Biosciences.

Aspects of the invention also relate to a cell, into which a nucleotide sequence coding for a recombinant protein of interest, as defined above, has been introduced. The sequence may be introduced in the form of a vector as defined above.

“Cell” will be understood to mean both a eukaryotic cell and a prokaryotic cell, particularly a bacterium. Any bacterium capable of allowing the expression of a protein from a nucleotide sequence may be used. By way of example, mention may be made of all bacteria which derive from BL21, BL21 star, Rosetta, BLR, Origami, Tuner, Novablue, all commercially available.

The method produces at least one protein of interest, characterized in that, in a first step, there is introduced into a cell a nucleotide sequence coding for a recombinant protein of interest, as defined above, and in that, in a second step, the cell is placed under conditions sufficient for allowing the expression of the recombinant protein of interest.

The method may further comprise an additional step during which the recombinant protein of interest may be cut by the action of an endoprotease (factor Xa, thrombin, for example), at the site created in the last residues of the integrin to separate the protein of interest from its fusion partner.

The method may also comprise an additional step during which the recombinant protein of interest, or the protein(s) of interest separated from its (their) fusion partner(s), may be purified.

The nucleotide sequence coding for a recombinant protein of interest may be introduced into the cell by any known method. By way of example of methods which can be used, it is possible to mention, in respect of prokaryotic cells, heat shock or electroporation. In respect of eukaryotic cells, mention may be made of electroporation, the calcium phosphate precipitate method, the use of cationic polymers such as DEAE-dextran, or any method using cationic liposomes or activated dendrimers. It is also possible to use retroviruses to carry out gene transfer, and also techniques using microprojectiles to deliver DNA to target cells.

Likewise, any sufficient condition known in the art which allows the expression of the recombinant protein of interest can be used according to the method.

Finally, any method of purifying the protein(s) which is known in the art can be used. By way of example, mention may be made of the methods of affinity chromatography, ion exchange chromatography, hydrophobic interaction chromatography or filtration using a molecular sieve.

In particular, when the recombinant protein of interest comprises the 6×HIS tag (SEQ ID NO: 12), purification on a nickel-nitrilotriacetic acid (Ni-NTA) agarose column represents one method of purification which is particularly satisfactory within the context of the method of the invention.

The techniques which can be used are known in the art. The latter can refer to the numerous manuals which are available, and in particular to “Molecular Cloning, a laboratory manual. 2nd edition, Sambrook, Fritsch, Maniatis eds., CSH laboratory press, (1989)”.

The following examples illustrate selected aspects of the invention and do not limit it in any way.

### EXAMPLE 1

### Construction of a Vector Which Allows the Expression in Bacteria of a Recombinant Protein of Interest

A complementary DNA coding for the protein of interest which it is desired to express is positioned in the vector pET21a (+) (sold by the company Novagen) in phase with a fragment of complementary DNA of α5-integrin, by using appropriate restriction sites. The α5-integrin fragment is delimited by the NdeI and BamHI sites. The NdeI site has the advantage of incorporating an ATG codon which is the translation initiator codon. This initiator codon codes for a methionine (M). The NdeI site formed by the sequence CATATG is thus of interest for subcloning a DNA fragment since the target sequence is located directly in phase with the ATG codon. The latter then forms residue 1. With regard to alpha5-integrin, the ATG of the NdeI site is positioned upstream of its nucleotide sequence. In this case, the ATG will code for an M1 and the G of the integrin will be residue 2. The fragment of 287 residues will be coupled to methionine 1 and a fusion partner of 288 residues is thus obtained: M1-G288.

The vector directly provides the sequence coding for the 6×HIS tag (SEQ ID NO: 12) which will be located at the C-terminal end of the recombinant protein of interest. An EcoRI site is located in the vector at the N-terminal end of the tag site. Thus, the complementary DNA coding for the protein of interest is inserted between the BamHI site marking the C-terminal end of the complementary DNA fragment of the α5-integrin and the EcoRI site located at the N-terminal end of the 6×HIS tag (SEQ ID NO: 12).

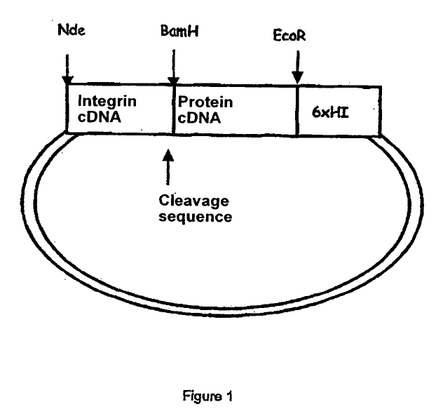

FIG. 1 shows the diagram of such a construct.

### EXAMPLE 2

### Expression of the Human Vasopressin V2 Receptor

Construction of the Vector:

The complementary DNA of the human vasopressin V2 receptor (Cotte et al., J. BIOL. Chem. 273, 29462-29468, 1998) is inserted between the BamHI and EcoRI sites of the vector obtained in Example 1.

Step 1: Preparation of the Complementary DNA of the Human Vasopressin V2 Receptor:

Recognition sites for the restriction enzymes BamHI and EcoRI are added on either side of the complementary DNA sequence of the human vasopressin V2 receptor. This is done using the conventional PCR technique. The complementary DNA of the human vasopressin V2 receptor is amplified from the vector pRK5-V2 (Cotte et al., J. BIOL. Chem. 273, 29462-29468, 1998) with the aid of two primer oligonucleotides which make it possible to insert the desired restriction sites:

| | |
|---|---|
| sense oligo (allows the | |
| incorporation of the BamHI site): | |
| 5′ ATG GGT CGC GGA TCC ATG CTC ATG | (SEQ ID NO: 13) |
| GCG TCC ACC ACT TCC 3′ | |
| antisense oligo (allows the | |
| incorporation of the EcoRI site): | |
| 5′ CGA CGG AAT TCT GCG ATG AAG TGT | (SEQ ID NO: 14) |
| CCT TGG CCA G 3′. | |

The PCR reaction is carried out in 50 microliters of a reaction mixture comprising:

| | | |
|---|---|---|
| pRK5-V2 | 20 | ng |
| sense oligo | 100 | ng |
| antisense oligo | 100 | ng |
| Pfu Turbo polymerase (Stratagene) | 2.5 | U |
| 10× Pfu buffer (Stratagene) | 5 | μl |
| dNTP 80 μM final for each of the 4 | | |

according to the following cycle parameters:
initial denaturation at 95° C. for 2 minutes, then
25 cycles: 95° C., 30 seconds then 55° C., 1 minute, 72° C., 1.5 minutes, then
final elongation at 72° C. for 10 minutes.

The presence of the amplified fragment (amplified PCR V2 fragment) is checked on 1% agarose gel.

Step 2: Purification of the Amplified Fragment (Amplified PCR V2 fragment):

The zone of the agarose gel in which the amplified DNA fragment is visualized is cut, and the cDNA is purified using the purification kit Qiaquick gel extraction kit (Qiagen reference 28706), adhering strictly to the protocol recommended by the supplier.

Step 3: Cutting of the Amplified PCR V2 Fragment by the Enzymes BanHI and EcoRI:

This is carried out in a single step using the enzymes sold by New England Biolabs (NEB) by incubation for 3 hours at 37° C., in a final volume of 50 microliters containing:

| | |
|---|---|
| V2 insert (amplified fragment) 100 to 200 ng | 24 μl |
| 10× EcoRI buffer NEB | 5 μl |
| bovine serum albumin NEB 100× (10 mg/ml) | 1 μl |
| EcoRI (40 U) | 2 μl |
| BamHI (40 U) | 2 μl |
| water | 16 μl |

At the end of the reaction, the two enzymes are inactivated by heating at 80° C. for 20 minutes.

The PCR V2 fragment is then purified using a 1% agarose gel according to the protocol described above.

Step 4: Subcloning of the Amplified PCR V2 Fragment in the BamHI and EcoRI Sites of the Vector pET21a of Example 1:

Ligation is carried out by incubating at ambient temperature (20-25° C.) for 4 hours in a medium comprising:

| | | |
|---|---|---|
| BamHI/EcoRI PCR V2 fragment (100 to 200 ng) | 8 | μl |
| vector pET21a (30 ng) cut by BamHI/EcoRI | 3 | μl |
| 10× ligase buffer (NEB) | 2.5 | μl |
| T4 DNA ligase (NEB) | 2 | μl |
| water | 9.5 | μl. |

The ligation product, the integrin/human vasopressin V2 receptor fusion protein, is then used for transformation of Rosetta bacteria (DE3) in order to carry out the receptor expression tests.

Introduction of the expression vector into a bacterium and expression of the protein of interest:

Transformation:

The vector obtained above is then introduced into a bacterium of the Rosetta strain (DE3) using the heat shock technique, following the transformation protocol recommended by the supplier, in this case Novagen.

20 μl of Rosetta bacteria (DE3), Novagen reference 70954-4, and 1 μl of pET21a-integrin/V2 (a few nanograms) are incubated on ice for 30 minutes, then kept at 42° C. for 30 seconds and then again on ice for 2 minutes in order to perform a heat shock.

80 μl of SOC medium (see composition in Molecular Cloning, a laboratory manual. 2nd edition, Sambrook, Fritsch, Maniatis eds. CSH laboratory press, 1989) are then added, then the whole is incubated at 37° C. for 1 hour with stirring at 300 rpm.

The incubation medium is then spread onto Petri dishes containing LB agar+ampicillin at 100 micrograms/ml. The dishes are incubated at 37° C. for 16 hours. The bacteria of a colony are then cultured at 37° C. in 10 ml of LB medium containing 100 μg/ml of ampicillin (or of its analog, carbenicillin), and the cell suspension is stirred at 300 rpm.

Expression of the Protein:

When the optical density of the culture reaches 0.6 U, expression of the recombinant protein is induced by adding 1 mM IPTG.

Samples are taken 2, 3 and 4 hours after induction. To do this, 1 ml of bacterial suspension, with an optical density of 0.6, is taken from each culture. The sample is centrifuged for 2 minutes at 12000 rpm. The supernatant is removed and the pellet is resuspended in 60 μl of lysis buffer (25 mM Tris, pH 8.3, 185 mM glycine, 0.1% SDS). 60 μl of SDS buffer (10% glycerol, 5% 2-mercaptoethanol, 25 mM Tris-HCl, pH 6.5, 8% SDS, bromophenol blue (a few grains)) are then added and 10 μl of the lyzed sample (total protein extracts) are then deposited on acrylamide/bis-acrylamide 12%-SDS 0.1% gel. Following migration of the proteins, the latter are stained with Coomassie blue according to conventional techniques.

FIG. 2 shows the results obtained. The induced samples are compared with controls which have not been induced (NI) but which have been cultured for an equivalent time. It can be seen that the α5-V2 receptor fusion protein, which has an apparent molecular weight of around 65 kDa, is one of the majority proteins of the bacterium, this being a condition which is necessary for purification of the receptor in a quantity compatible with analyses of its structure using NMR or crystallography approaches.

1 ml of culture thus formed made it possible to obtain around 3 μg of vasopressin receptor.

### EXAMPLE 3

### Expression of Other Receptors

The result obtained in Example 2 was reproduced with the same effectiveness for other GPCRs, such as the α3-adrenergic receptor, the BLT2, Cys-LT1 and Cys-LT2 receptors of leukotrienes LTB4, LTD4 and LTC4, the cannabinoid receptor type 1, the vasopressin V1a receptor and the oxytocin receptor.

### EXAMPLE 4

### Purification of the α5-integrin Fragment-vasopressin V2 Receptor Fusion Protein Obtained in Example 2

The method used is that described by Porath J. et coll. (Metal chelate affinity chromatography, a new approach to protein fractionation. Nature 258, 598-599, 1975).

The example described here is a test which made it possible to purify 3 mg of fusion protein from a bacterial culture of 100 ml.

A colony isolated on LB agar+ampicillin (100 μg/ml) is pricked and cultured in 10 ml of culture medium LB+carbenicillin (100 μg/ml). Culturing is carried out at 37° C., with stirring at 300 rpm. When the optical density of the culture reaches 0.6, culturing is stopped and the culture is kept in the refrigerator (this sample is called the “preculture”). The next day, in a 500 ml Erlenmeyer, 100 ml of culture medium LB+carbenicillin (100 μg/ml) are seeded with 2 ml of preculture and left at 37° C., at 300 rpm, until the optical density of the culture has reached 0.6. 0.1 mM IPTG is then added to the culture to induce expression of the recombinant protein. Culturing is continued for around 3 hours, until an optical density of 2.4 is obtained (stimulation factor of 4).

The culture is then centrifuged at 4000 rpm for 5 minutes. The supernatant is removed and the pellet can be lyzed directly or kept at −80° C.

For lysis, the pellet is taken up by homogenization using a pipette in 6 ml of Tris-HCl 20 mM, pH 8.00+protease inhibitors (leupeptin 5 μg/ml; benzamidine 10 μg/ml and PMSF 10 μg/ml). These three protease inhibitors will be incorporated in all the buffers used hereafter.

The bacteria are lyzed by sonication using a Branson conical microprobe (duty cycle 50%, output control 5, frequency 1 burst per second for 30 seconds, then rest for 30 seconds; this cycle is repeated 5 times). The tube is kept in ice during the sonication. The medium is then centrifuged for 30 minutes at 15000 rpm at 4° C. The supernatant is kept for control on electrophoresis gel.

The pellet contains the protein of interest since the latter has accumulated in the inclusion body.

The pellet is taken up by homogenization using a pipette in 5 ml of Tris-HCI 20 mM, pH 8.00. The lysis and centrifugation steps are repeated once.

The centrifugation supernatants are kept for control on electrophoresis gel.

The pellet is taken up by homogenization using a pipette in 5 ml of Tris-HCl, pH 8.00, lM urea. A magnetic bar is placed in the sample and the latter is stirred gently for 1h30. The tube is kept in ice during this step, which corresponds to washing of the inclusion body and makes it possible to remove membrane proteins or cytoplasmic proteins which are associated with the inclusion bodies but which are considered as contaminants with respect to the recombinant protein.

The whole is then centrifuged at 15000 rpm for 30 minutes at 4° C. The supernatant is kept for control on electrophoresis gel.

The pellet is taken up by homogenization using a pipette in 5 ml of Tris-HCI 20 mM, pH 8.00, 6M urea, SDS 0.2% to solubilize the inclusion bodies and thus the protein of interest.

A magnetic bar is placed in the sample and the latter is stirred gently for 3 hours, in ice.

The protein of interest (the α5-integrin fragment-vasopressin V2 receptor fusion protein) is then completely denatured.

The whole is then centrifuged at 15000 rpm for 30 minutes at 4° C. The supernatant contains the protein of interest and constitutes the sample which will be brought into contact with the Ni-NTA (nickel-nitrilotriacetic acid) resin to purify the alpha5-V2 fusion by means of affinity chromatography.

3 ml of Superflow Ni-NTA agarose resin (Qiagen, ref. 30430) are equilibrated in Tris-HCI 20 mM, pH 8.00, 6M urea, SDS 0.2%, NaCl 150 mM, imidazole 5 mM. A sufficient amount of NaCl and imidazole are added to the sample containing the protein of interest to obtain a final concentration of 150 mM of NaCl and 5 mM of imidazole. The sample and the resin are brought into contact and left to incubate at 4° C. for 16 hours with gentle stirring. The sample/resin mixture is deposited in a plastic column and left. After settling, the “flow-through” fraction is recovered at a low flow rate, for control on electrophoresis gel.

The resin is then washed with 3×9 ml of a solution of Tris-HCl 20 mM, pH 8.00, 6M urea, SDS 0.2%, NaCl 150 mM, imidazole 20 mM, to remove all the proteins not specifically held on the nickel groups. The wash eluates are kept for control on electrophoresis gel.

The protein of interest is then detached from the resin by passing through 3 ml of a solution of Tris-HCl 20 mM, pH 8.00, 6M urea, SDS 0.2%, NaCl 150 mM, imidazole 100 mM. An aliquot of the purified protein is kept for control on electrophoresis gel.

10 μl of the medium containing the purified protein are mixed with 10 μl of SDS buffer and the whole is deposited on an electrophoresis gel.

10 μl of the purified sample contain from 5 to 10 μg of protein, that is to say that 1.5 to 3 mg of recombinant protein are contained in 3 ml of eluate.

FIG. 3 shows the purified protein deposited on electrophoresis gel.

The purified sample is dialyzed against a solution of Tris-HCl 20 mM, pH 8.00, 6M urea, NaCl 150 mM to remove the SDS and the imidazole. To do this, the sample is placed in a Pierce dialysis cassette (membrane of 10000 MWCO) and dialysis is carried out in a beaker containing one liter of buffer. The dialysis is carried out at 4° C. for at least 24 hours.

The sample is recovered and the amount of protein of interest which is obtained is metered by measuring the absorption (excitation at 280 nM, absorption between 235 and 500 nM). In general, an O.D. of 1 to 1.5 is obtained, which is equivalent to a concentration of 0.5 to 1 mg/ml, which is equivalent to a concentration of around 10 μM.

The protein which has been purified and denatured (since it has been solubilized in 6M urea) is used for the renaturation tests.

### EXAMPLE 5

### Construction of a Vector Which Allows the Simultaneous Expression of Two Proteins of Interest (in this Case Two GPCRs) in Escherichia coli and Their Purification

A complementary DNA coding for a protein of interest, in this case the human chemokine receptor CXCR4, is inserted in the vector pET21a(+)−α5V2 described above in Example 2. This DNA must be in phase with that coding for the α5V2 fusion and is positioned between the SacI and HindIII restriction sites for example. The vector directly supplies the sequence coding for the 6×HIS tag (SEQ ID NO: 12) which will thus be located at the C-terminal end of the receptor CXCR4 and will therefore allow its purification in a subsequent step.

In the example, an optimized (“bacterialized”) version of the CXCR4 is inserted in the vector, but the natural eukaryotic version of this receptor (Herzog H, Hort Y J, Shine J and Selbie L A. Molecular cloning, characterization and localization of the human homolog to the reported bovine NPY Y3 receptor: lack of NPY binding and activation. DNA Cell Biol. 12, 465-471, 1993) can also be used in the same way.

Step 1: Preparation of the Complementary DNA of the Human Receptor CXCR4.

Recognition sites for the restriction enzymes SacI and HindIII are added on either side of the sequence coding for the human receptor CXCR4 during a conventional PCR reaction. The complementary DNA of this receptor is amplified from the vector pET101/D-TOPO (Invitrogen) in which it is subcloned and from two primer oligonucleotides which make it possible to insert the restriction sites in question.

| | |
|---|---|
| Sense oligo (incorporation of the | |
| SacI site): | |
| 5′ CGAGCTAAGGC GAGCTC A | (SEQ ID NO: 15) |
| ATGGAAGGCATTAGCATTTATAC 3′ | |
| Antisense oligo (incorporation of | |
| the HindIII site): | |
| 5′ CGACGGCCC AAGCTT | (SEQ ID NO: 16) |
| GCTGCTATGAAAGCTGCTGCTTTC 3′. | |

The PCR reaction is carried out in 50 microliters and is composed of:

| | | |
|---|---|---|
| pET101/D-TOPO | 20 | ng |
| sense oligo | 100 | ng |
| antisense oligo | 100 | ng |
| Pfu Turbo polymerase (Stratagene) | 2.5 | U |
| 10× Pfu buffer | 5 | μl |
| dNTP 80 μM final for each of the 4 | | |

The reaction parameters are:
initial denaturation at 95° C. for 2 minutes, then 25 cycles: 95° C., 30 s; 55° C., 1 min; 72° C., 1.5 min, then final elongation at 72° C., 10 min.

The presence of the amplified fragment is checked on 1% agarose gel.

Step 2: Purification of the Amplified Fragment

The zone of the agarose gel in which the amplified DNA fragment is visualized is cut, and the cDNA is purified using the purification kit Qiaquick gel extraction kit (Qiagen reference 28706), adhering strictly to the protocol recommended by the supplier.

Step 3: Cutting of the Amplified PCR CXCR4 Fragment by SacI and HindIII:

This is carried out in two successive reactions using restriction enzymes sold by NEB Biolabs during incubation at 37° C.

1st reaction for 3h:

| | | |
|---|---|---|
| PCR fragment | 500 | ng |
| 10× NEB buffer 1 | 5 | μl |
| bovine serum albumin (BSA) | 1 | μl |
| SacI | 40 | U |
| water qs | 50 | μl |

The PCR fragment which has been amplified and cut by SacI is purified using an agarose gel according to the protocol described in step 2.

2nd reaction for 3h:

PCR fragment recovered from the previous reaction

| | | |
|---|---|---|
| 10× NEB buffer 2 | 5 | μl |
| BSA | 1 | μl |
| HindIII | 4 | U |
| water qs | 50 | μl |

The PCR fragment which has been amplified and cut by SacI/HindIII is purified using an agarose gel according to the protocol described in step 2.

Step 4: Subcloning of the PCR CXCR4 Fragment Which Has Been Amplified and Cut by SacI/HindIII in the vector pET21a(+)−α5V2:

This step is carried out by ligation at ambient temperature for 16h00.

| | | |
|---|---|---|
| cut PCR fragment | 200 | ng |
| vector pET21a(+)-α5V2 cut by the same enzymes | 50 | ng |
| 10× ligase buffer (NEB) | 2.5 | μl |
| T4 DNA ligase (NEB) | 2 | μl |
| water qs | 25 | μl. |

The vector obtained, which codes for a triple fusion protein, integrin-V2 receptor-CXCR4 receptor, is then used for a transformation of Rosetta bacteria (DE3) for the purpose of expressing this fusion and purifying it.

Step 5: Transformation of the Rosetta Bacteria

Follow the protocol described in Example 2.

Step 6: Expression of the α5V2-CXCR4 Fusion

Follow the protocol described in Example 2, but the LB culture medium is replaced by Hyperbroth medium (Athena Enzyme Systems) and the optimal induction time is 4 hours.

Step 7: Purification of the α5V2-CXCR4 Fusion. This Step is Shown in FIG. 4.

Follow the protocol of Example 4, but, during the step of washing the Ni-NTA agarose resin, a concentration of 15 mM of imidazole instead of 20 mM is used in the wash solution. Elution is carried out to 100 mM as in Example 4.

The spacer arm can also be inserted upstream of the thrombin cleavage site just after the EcoRI site.

1-30. (canceled)

31. A recombinant protein comprising a fragment of an alpha-integrin for producing at least one recombinant protein of interest in a cell, with the exception of a mammalian cell.

32. The recombinant protein of claim 31, wherein the cell is a bacterium.

33. The recombinant protein of claim 31, which is a complete amino acid sequence of the alpha-integrin or a partial sequence.

34. The recombinant protein of claim 31, which is a sequence comprising the N-terminal end of the alpha-integrin used.

35. The recombinant protein of claim 31, which is native or mutated.

36. The recombinant protein of claim 31, comprising at least FG-GAP modules IV to VII and a portion of FG-GAP module III of the alpha-integrin.

37. The recombinant protein of claim 31, originating from at least one of an alpha-integrin selected from the group consisting of the integrins α1, α2, α3, α4, α5, α6, α7, α8, α9, α10, α11, αD, αE, αL, αM, αX, αIIb and αV.

38. The recombinant protein of claim 37, originating from at least one of an alpha-integrin selected from the group consisting of the integrins α5, αV and αIIb.

39. The recombinant protein of claim 38, wherein the alpha-5-integrin fragment extends between positions 231 and 517 (taking account of the presence of the signal peptide) or positions 190 to 476 (not taking account of the presence of the signal peptide).

40. The recombinant protein of claim 38, wherein the αV-integrin fragment extends between positions 211 and 495 (taking account of the presence of the signal peptide) or positions 181 to 465 (not taking account of the presence of the signal peptide).

41. The recombinant protein of claim 38, wherein the αIIb-integrin fragment extends between positions 224 and 508 (taking account of the presence of the signal peptide) or positions 193 (G residue) to 477 (Q residue) of αIIb-integrin.

42. The recombinant protein of claim 31, comprising at least one amino acid sequence selected from the group consisting of SEQ ID No. 1, SEQ ID No. 2 and SEQ ID No. 3.

43. The recombinant protein of claim 31, comprising at least one amino acid sequence encoded by one of the nucleotide sequences selected from the group consisting of SEQ ID No. 4, SEQ ID No. 5 and SEQ ID No. 6.

44. The recombinant protein of claim 31, located in the recombinant protein(s) of interest upstream of sequence(s) of the protein(s) of interest to be produced.

45. The recombinant protein of claim 31, wherein the recombinant protein(s) comprises(comprise) at least one endoprotease cleavage site.

46. The recombinant protein of claim 31, wherein the recombinant protein(s) comprises(comprise) at least one spacer arm.

47. The recombinant protein of claim 46, wherein the spacer arm consists of peptide sequence SEQ ID No. 8.

48. The recombinant protein of claim 46, wherein the recombinant protein comprises at least one spacer arm encoded by nucleic acid sequence SEQ ID No. 7.

49. The recombinant protein of claim 31, wherein the protein of interest is a G protein-coupled receptor.

50. The recombinant protein of claim 49, wherein the G protein-coupled receptor is at least one selected from vasopressin and oxytocin receptors (V1a, V2, OTR), leukotriene receptors (BLT1, BLT2, CysLT1, CysLT2), adrenergic receptors (beta-3), cannabinodi receptors (CB1), chemokine receptors (CCR5, CXCR4), the angiotensin II AT1 receptor, and the bradykinin B2 receptor.

51. The recombinant protein of claim 31, comprising at least one sequence of 6 histidine residues (6×HIS tag; SEQ ID NO: 12)).

52. The recombinant protein of claim 51, wherein the sequence of 6 histidine residues is at the C-terminal end of the protein.

53. A nucleotide sequence coding for at least one recombinant protein of interest as described in claim 31.

54. A vector comprising a nucleotide sequence of claim 53.

55. A cell, with the exception of a mammalian cell, into which a nucleotide sequence of claim 53 or a vector as described in claim 54 has been introduced.

56. A method for producing at least one protein of interest comprising:
introducing into a cell, with the exception of a mammalian cell, a nucleotide sequence coding for at least one recombinant protein of claim 53, and
placing the cell under conditions which allow expression of the recombinant protein(s) of interest.

57. The method as claimed in claim 56, further comprising cutting the recombinant protein(s) of interest with an endoprotease.

58. The method of claim 56, further comprising purifying the recombinant protein(s) of interest, or the protein(s) of interest separated from its(their) fusion partner(s).


SEQUENCE LISTING (16 sequences)

SEQ ID NO: 1 - protein, 288 aa, Homo sapiens
MGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEG

SEQ ID NO: 2 - protein, 286 aa, Homo sapiens
MGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPG

SEQ ID NO: 3 - protein, 286 aa, Homo sapiens
MGLLAQAPVADIFSSYRPGILLWHVSSQSLSFDSSNPEYFDGYWGYSVAVGEFDGDLNTTEYVVGAPTWSWTLGAVEILDSYYQRLHRLRAEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQ

SEQ ID NO: 4 - DNA, 864 bp, Homo sapiens
atgggccagatcctgtctgccactcaggagcagattgcagaatcttattaccccgagtacctgatcaacctggttcaggggcagctgcagactcgccaggccagttccatctatgatgacagctacctaggatactctgtggctgttggtgaattcagtggtgatgacacagaagactttgttgctggtgtgcccaaagggaacctcacttacggctatgtcaccatccttaatggctcagacattcgatccctctacaacttctcaggggaacagatggcctcctactttggctatgcagtggccgccacagacgtcaatggggacgggctggatgacttgctggtgggggcacccctgctcatggatcggacccctgacgggcggcctcaggaggtgggcagggtctacgtctacctgcagcacccagccggcatagagcccacgcccacccttaccctcactggccatgatgagtttggccgatttggcagctccttgacccccctgggggacctggaccaggatggctacaatgatgtggccatcggggctccctttggtggggagacccagcagggagtagtgtttgtatttcctgggggcccaggagggctgggctctaagccttcccaggttctgcagcccctgtgggcagccagccacaccccagacttctttggctctgcccttcgaggaggccgagacctggatggcaatggatatcctgatctgattgtggggtcctttggtgtggacaaggctgtggtatacaggggccgccccatcgtgtccgctagtgcctccctcaccatcttccccgccatgttcaacccagaggagcggagctgcagcttagagggg

SEQ ID NO: 5 - DNA, 858 bp, Homo sapiens
atgggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatgatattgcaattgctgctccatatgggggtgaagataaaaaaggaattgtttatatcttcaatggaagatcaacaggcttgaacgcagtcccatctcaaatccttgaagggcagtgggctgctcgaagcatgccaccaagctttggctattcaatgaaaggagccacagatatagacaaaaatggatatccagacttaattgtaggagcttttggtgtagatcgagctatcttatacagggccagaccagttatcactgtaaatgctggtcttgaagtgtaccctagcattttaaatcaagacaataaaacctgctcactgcctgga

SEQ ID NO: 6 - DNA, 858 bp, Homo sapiens
atgggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcgcagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcag

SEQ ID NO: 7 - DNA, 33 bp, Artificial Sequence
  Description of Artificial Sequence Synthetic nucleotide sequence
gacccgggtggtggtggtggtggtggtggtggt

SEQ ID NO: 8 - protein, 10 aa, Artificial Sequence
  Description of Artificial Sequence Synthetic peptide
DPGGGGGGGG

SEQ ID NO: 9 - protein, 4 aa, Artificial Sequence
  Description of Artificial Sequence Synthetic peptide
IEGR

SEQ ID NO: 10 - protein, 4 aa, Artificial Sequence
  Description of Artificial Sequence Synthetic peptide
LVPR

SEQ ID NO: 11 - protein, 6 aa, Artificial Sequence
  Description of Artificial Sequence Synthetic peptide
LVPRGS

SEQ ID NO: 12 - protein, 6 aa, Artificial Sequence
  Description of Artificial Sequence Synthetic 6xHis tag
HHHHHH

SEQ ID NO: 13 - DNA, 39 bp, Artificial Sequence
  Description of Artificial Sequence Synthetic primer
atgggtcgcggatccatgctcatggcgtccaccacttcc

SEQ ID NO: 14 - DNA, 34 bp, Artificial Sequence
  Description of Artificial Sequence Synthetic primer
cgacggaattctgcgatgaagtgtccttggccag

SEQ ID NO: 15 - DNA, 41 bp, Artificial Sequence
  Description of Artificial Sequence Synthetic primer
cgagctaaggcgagctcaatggaaggcattagcatttatac

SEQ ID NO: 16 - DNA, 39 bp, Artificial Sequence
  Description of Artificial Sequence Synthetic primer
cgacggcccaagcttgctgctatgaaagctgctgctttc